(19) United States
(12) Patent Application Publication
(10) Pub. No.: US 2006/0234327 A1
(43) Pub. Date: Oct. 19, 2006

(54) Methods for identifying enzyme inhibitors and protein kinases

(76) Inventors: Harold Neal Bramson, King of Prussia, PA (US); George I. Glover, King of Prussia, PA (US); Sanjay Khandekar, King of Prussia, PA (US); Steven John Ratcliffe, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/549,390

(22) PCT Filed: Mar. 17, 2004
(86) PCT No.: PCT/US04/08043

§ 371 Date: Sep. 14, 2005

Related U.S. Application Data
(60) Provisional application No. 60/455,374, filed on Mar. 17, 2003.
(60) Provisional application No. 60/487,983, filed on Jul. 17, 2003.

(51) Int. Cl. C12Q 1/48

(52) U.S. Cl. 435/15; 435/183

(57) ABSTRACT

The present invention provides methods for identifying compounds that inhibit kinases. In addition, methods for profiling protein kinases are also provided. In addition, methods for determining the mode of action of kinase inhibitors are also provided.

### BACKGROUND OF THE INVENTION

Protein kinases represent one of the largest protein super-families in eukaryotes. Based on functional classification of human genes, the number of protein kinases encoded in the human genome is estimated to be in excess of 500 but less than 1000, which comprise about 3% of the total human genes (Venter, et al., Science, 291:1304-1351(2001)). Protein kinases play important roles in signal transduction pathways and in many cell regulatory processes such as cell division and differentiation, development, oncogenesis, cell survival and apoptosis. In addition, the genes encoding many protein kinases are located upstream or downstream of many of the epidemiologically relevant genes, underscoring them as targets for therapeutic intervention. It is therefore not surprising that protein kinases have emerged as attractive targets for identification and development of novel therapeutic agents for many disease indications.

Sequence alignment studies have revealed that most protein kinases share a common core of about 270 amino acids (Hank, S. K., and Hunter, T., The Protein Kinase Facts Book, Ed G. Kardie and S. Hanks, Academic Press, NY. (1995)). Studies comparing crystal structures of kinases have shown that the core structure of all kinases adopts a common fold (Hanks and Hunter, 1995). Most noteworthy, the regions in the N-terminal domain involved in ATP binding and the C-terminal domain involved in protein substrate recognition adopt similar conformation in most kinases as revealed by their crystal structures in their active forms. The ATP binding site is situated at the interface of the N-terminal and C-terminal lobes.

Structural studies shown that most of the small molecule kinase inhibitors studied so far bind to the Mg-ATP complex binding pocket (Garcia-Echeverria et al., Med. Res. Rev., 20:28-57 (2000)). As a result, considerable progress has been made towards the synthesis of potent and selective ATP site directed protein kinase inhibitors by modulating and fine-tuning the chemical templates (Garcia-Echeverria et al., 2000). Together these studies have refuted the accepted idea that the ATP binding cleft is not an ideal target for development of potent, specific inhibitors.

p-Flurosulfonylbenzoyl 5′-adenosine (FSBA) is an ATP-affinity reagent that is effective in covalent modification of nucleotide-binding sites in a variety of protein kinases (Kamps, et al., Nature 310:589-592 (1984); Scoggins, et al., Biochemistry 35:9197-9203 (1996); Young, et al., J. Biol. Chem. 272: 12116-12121 (1997); Fox, et al., FEBS Letts. 461:323-328 (1999)). Structurally FSBA is similar to ATP except for the presence of flurosulfonylbenzoyl moiety in place of the three phosphates of ATP. FSBA has been used to selectively label and characterize a number of protein kinases. Peptide mass finger printing studies carried out on a panel of protein kinases have shown that FSBA binds irreversibly to the side chain of a critical, conserved lysine found in the ATP binding site (Kamps, et al., 1984; Zoller et al., J. Biol. Chem. 256:10837-10842 (1981)). ¹⁴C-labeled FSBA (Fox, et al., 1999; Buhrow, et al., . Biol. Chem. 258:7824-7827 (1983)) and FSBA-specific antibodies (Parker, FEBS Letts. 334:347-350 (1993); T'Jampens, et al., FEBS Letts. 516:20-26 (2002)) have been used to identify and characterize protein kinases from cell lysates, although these reagents because of their limitations have not been effectively used for inhibitor screening (T'Jampens, et al., 2002).

Liquid chromatography/mass spectrometry (LC/MS) technique has been valuable in monitoring covalent modification of natural and recombinantly expressed proteins (Feng, et al., Anal. Chem. 73:5691-5697 (2001)). These techniques have also been routinely used in drug metabolism and pharmacokinetics studies (Feng, et al., 2001), but their use as screening tools has not been widely explored.

Accordingly, a method for identifying compounds that inhibit kinases is greatly needed. In addition, a method for profiling protein kinases is also greatly needed.

### SUMMARY OF THE INVENTION

One embodiment of the present invention is to provide a method for identifying a compound that inhibits an enzyme having an ATP binding site comprising contacting a composition of the enzyme, an analyte capable of binding to the ATP binding site of the enzyme, and a test compound, and detecting whether the test compound inhibits the analyte from binding the ATP binding site of the enzyme.

A further embodiment of the present invention, provides a method for identifying a compound that inhibits a kinase having an ATP binding site comprising the steps of contacting a composition comprising the kinase and an analyte that binds to an ATP binding site of the kinase; detecting binding of the analyte to the ATP binding site; contacting a composition comprising the kinase, the analyte, and a test compound, and detecting whether the test compound inhibits the analyte from binding the ATP binding site.

In another embodiment of the present invention, a method is provided for identifying a test compound that inhibits a kinase with an ATP-binding site comprising the steps of contacting a composition comprising the kinase and test compound; contacting the composition comprising the kinase and the test compound with an analyte; and detecting whether the test compound inhibits the analyte from binding the kinase's ATP binding site.

Also provided in the instant invention is a method wherein detecting the binding of the test compound to the kinase comprises using liquid chromatography/mass spectrometry.

In another embodiment of the present invention, a method is provided for identifying a protein kinase having an ATP binding site comprising the steps of: contacting a composition comprising the protein kinase with an analyte capable of binding said kinase, and detecting whether analyte binds to said kinase.

In another embodiment, biotin-FSBA is provided.

Also provided in the present invention is a method for identifying the mode of action of a test compound that inhibits a first enzyme having a first ATP binding site comprising the steps of: (a) contacting a composition comprising the first enzyme having a first ATP binding site, an analyte capable of binding to the first ATP binding site of said enzyme, and a test compound, wherein the ratio of concentration of analyte to test compound is at least 1:1; and (b) detecting whether said test compound inhibits said analyte from binding said first ATP binding site, wherein diminution of analyte binding to the first ATP binding site indicates competitive inhibition by said test compound. In another aspect of the invention, a method is provided for identifying the mode of action of the test compound comprising: (a) contacting a composition comprising a second enzyme having a second ATP binding site, an analyte capable of binding to the second ATP binding site of said second enzyme, and the test compound; (b) detecting whether said test compound inhibits said an analyte from binding said second ATP binding site; and (c) determining if said test compound is a selective inhibitor of said enzyme. The analyte used in each method may be the same or different. Also provided by the present invention is a method for identifying the mode of action of a compound wherein said test compound that diminishes binding of said analyte to said first enzyme but does not diminish binding of said an analyte to said second enzyme is a selective inhibitor of said first enzyme.

### DETAILED DESCRIPTION OF THE INVENTION

Glossary

As used herein “polypeptide” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may comprise amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may comprise many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2nd Ed., T. E. Creighton, W. H. Freeman and Company, New York (1993) and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

As used herein “polynucleotide(s)” generally refers to any polyribonucleotide or polydeoxyribonucleotide, that may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that comprise one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

As used herein “kinase” refers to any polypeptide capable of phosphorylating or adding a phosphate group to another molecule. The other molecule may be but is not limited to another polypeptide, a polynucleotide, or a cofactor. The kinase may be an enzyme capable of catalysis.

As used herein “biotin-” or “biotinylated” refers to any molecule having a biotin moiety attached to it. The biotin moiety may be attached covalently.

As used herein “protease assay” refers to an assay wherein the protease activity of a polypeptide is measured. Protease activity may include, but is not limited to; an enzymes ability to cleave a polypeptide into peptide fragments.

As used herein “kinase assay” refers to an assay wherein the activity of a kinase is measured. Kinase activity may include, but is not limited to, the rate or amount of phosphorylation of a substrate by a kinase.

As used herein “competitive inhibitor” refers to any compound that reduces the rate of enzyme catalysis by reducing the proportion of enzymes bound to substrate, by competing for the same binding site as a naturally occurring substrate. For instance, a competitive inhibitor may compete with the natural substrate of an enzyme by binding to an enzyme's active site.

As used herein “noncompetitive inhibitor” refers to a compound that has no effect on substrate binding to an enzyme. Substrate and inhibitor bind reversibly, randomly and independently at different sites on the enzyme.

As used herein “uncompetitive inhibitor” refers to a compound that does not bind free enzyme, but binds reversibly to the enzyme-substrate complex yielding inactive enzyme-substrate-inhibitor complex.

Recently flurophosphonate/flurophosphate (FP) derivatives were used in profiling serine hydrolases (Liu et al., Proc. Natl. Acad. Sci. USA 96:14694-14699(1999); Kidd et al., Biochemistry 40, 4005-4015 (2001)). The FP inhibitors display reactivity against a majority of serine hydrolases in an activity-dependent manner. FSBA reacts with a panel of kinases by binding to active sites, and its labeling appears to be dependent on the conformational/activity state of the kinase. Therefore, FSBA was used as an ABP for selecting ATP competitive inhibitors of kinases.

Although the radioactive form of FSBA, which is commercially available, can be used for kinase profiling studies, its detection by fluorography requires several days to weeks; In addition, handling and disposal of radioactive material also makes it a less attractive reagent for screening studies. Since FSBA modification of kinases is covalent, which is expected to be stable to LC/MS, LC/MS was used as part of this invention as an alternative to autoradiography.

Because FSBA binds covalently in the ATP binding pocket of protein kinases, LC/MS has utility in screening ATP competitive protein kinase inhibitors. This invention demonstrates that using purified recombinant kinases and FSBA as an activity-based probe (ABP), LC/MS provides a general, rapid and reproducible means to screen ATP competitor inhibitors of protein kinases.

In the present invention, autoradiography and LC/MS techniques are used to evaluate FSBA as an activity-based probe for protein kinases. The work presented here demonstrates FSBA's utility as an activity-based probe and LC/MS's usefulness as a screening tool for the selection of ATP competitor protein kinase inhibitors. Compared to fluorography, which takes days to weeks for evaluation, LC/MS allows rapid detection (e.g., within minutes) of the inhibitor screening. Furthermore, with the advent of a new integrated, 10-pump, eight channel, parallel LC/MS (Feng et al., 2001), this method has potential to be used in a high throughput mode as well.

Biotin may be used in conjunction with a visible marker. It is understood in the art that biotin can be detected with avidin or streptavidin linked to a molecule capable of providing a visible signal. Typically, avidin or streptavidin linked horseradish peroxidase is used in Western blot to detect biotinylated antibodies. As part of this invention, biotin has been covalently attached to FSBA which may then be detected by avidin or streptavidin linked to a marker. As used herein “Western blot” includes a method of detecting a polypeptide attached to a solid support wherein the polypeptide is contacted with a molecule having a biotin moiety. This biotin moiety may be attached to an antibody or to FSBA. The biotin moiety is then visualized using avidin or streptavidin attached to a molecule capable of providing a visual signal.

Thus, one embodiment of the present invention is to provide a method for identifying a compound that inhibits an enzyme having an ATP binding site comprising contacting a composition of the enzyme, an analyte capable of binding to the ATP binding site of the enzyme, and a test compound, and detecting whether the test compound inhibits the analyte from binding the ATP binding site. In one aspect of the invention, the enzyme is a kinase. In another aspect of the invention, the test compound is a competitive inhibitor of the analyte. In yet another aspect of the invention, the analyte is p-flurosulfonylbenzoyl 5′-adenosine (FSBA). In yet another aspect of the invention, the enzyme comprises a conserved lysine in the ATP binding site. In yet another aspect of the invention, the analyte is bound to the conserved lysine. Detecting whether the test compound inhibits the analyte from binding to the enzyme can be done by mass spectrometry, a protease assay, or a kinase assay.

In another embodiment of the present invention, a method is provided for identifying a compound that inhibits a kinase having an ATP binding site comprising the steps of contacting a composition comprising the kinase and an analyte that binds to an ATP binding site of the kinase; detecting binding of the analyte to the ATP binding site; contacting a composition comprising the kinase, the analyte, and a test compound, and detecting whether the test compound inhibits the analyte from binding the ATP binding site. In another aspect of the invention, the enzyme has a conserved lysine in the ATP binding site. In yet another aspect of the invention, the analyte is bound to the conserved lysine. In another aspect of the invention, the binding of the analyte to the ATP binding site can be performed by mass spectrometry, a protease assay or a kinase assay. In another aspect of the invention, the test compound is a competitive inhibitor of the analyte. The analyte may be p-flurosulfonylbenzoyl 5′-adenosine (FSBA).

In another embodiment of the present invention, a method is provided for identifying a test compound that inhibits a kinase having an ATP binding site comprising the steps of contacting a composition comprising the kinase and test compound; contacting the composition comprising the kinase and the test compound with an analyte; and detecting whether the test compound inhibits the analyte from binding the kinase's ATP binding site. In another aspect of the invention, the enzyme has a conserved lysine in the ATP binding site. In another aspect of the invention, the analyte is bound to the conserved lysine. Detection of whether the test compound inhibits the analyte from binding the kinase's ATP binding site can be done by using mass spectrometry, a protease assay, or a kinase assay. In yet another aspect of the invention, the test compound is a competitive inhibitor of the analyte. The analyte may be p-flurosulfonylbenzoyl 5′-adenosine (FSBA).

In yet another embodiment, a method is provided for identifying a protein kinase having an ATP binding site comprising the steps of contacting a composition comprising the protein kinase with an analyte capable of bind said kinase, and detecting whether analyte binds to said kinase. In another aspect, the analyte is p-flurosulfonylbenzoyl 5′-adenosine (FSBA). In another aspect, the protein kinase is contacted with a kinase inhibitor. In another aspect, Western blot is used to detect FSBA binding. In another aspect, LC/MS is used to detect FSBA binding.

In yet another embodiment, biotin-FSBA is provided. In another aspect the compound of Formula I is provided:

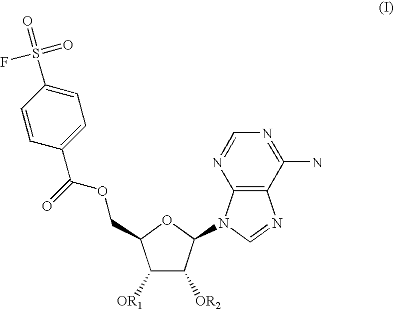

where
R₁ and R₂ are independently H or biotin as represented by Formula II:

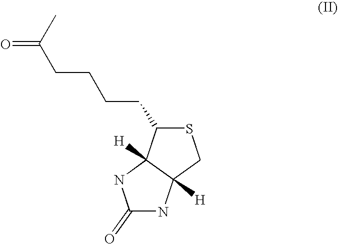

Biotinylated FSBA may be produced as a free compound for use in solution or it may be attached to a solid support or resin. In another embodiment, process for making a compound of Formula I is provided.

Also provided in the present invention is a method for identifying the mode of action of a test compound that inhibits a first enzyme having a first ATP binding site comprising the steps of: (a) contacting a composition comprising the first enzyme having a first ATP binding site, an analyte capable of binding to the first ATP binding site of said enzyme, and a test compound, wherein the ratio of concentration of analyte to test compound is at least 1:1; and (b) detecting whether said test compound inhibits said analyte from binding said first ATP binding site, wherein diminution of analyte binding to the first ATP binding site indicates competitive inhibition by said test compound. In another aspect of the invention, a method is provided for identifying the mode of action of the test compound comprising: (a) contacting a composition comprising a second enzyme having a second ATP binding site, an analyte capable of binding to the second ATP binding site of said second enzyme, and the test compound; (b) detecting whether said test compound inhibits said an analyte from binding said second ATP binding site; and (c) determining if said test compound is a selective inhibitor of said enzyme. The analyte used in each method may be the same or different. Also provided by the present invention is a method for identifying the mode of action of a compound wherein said test compound that diminishes binding of said analyte to said first enzyme but does not diminish binding of said an analyte to said second enzyme is a selective inhibitor of said first enzyme.

The following examples illustrate various aspects of this invention. These examples do not limit the scope of this invention, which is defined by the appended claims.

### EXAMPLES

Purified protein kinases were used in the following examples. Unless otherwise indicated, proteins and chemicals were obtained from Sigma. ¹⁴C-labeled FSBA was obtained from Perkin Elmer Life Sciences. Autoradiography on ¹⁴C-FSBA labeled protein kinases was carried out as described (Buhrow et al., 1983; Fox et al., 1997). The dried gels were exposed to film at −80° C. for 2 to 4 weeks. MS analysis of purified proteins and FSBA-modified protein kinases were carried out as described (Feng, et al., 2001).

For FSBA labeling, purified proteins (about 0.2 to about 0.5 mg/mL) were incubated with 10 μM FSBA (in 2.5% DMSO) at room temperature unless otherwise indicated. For time dependent modification studies, 10 μl aliquot was removed at an indicated time and was mixed with 50 μl of 0.1% TFA and injected on LC/MS as described before (Feng et al., 2001). For ATP protection experiments, purified kinases were coincubated with 10 μM FSBA and various amounts of ATP and MgCl₂ for 1 to 2 hours at room temperature. For staurosporine protection experiments, purified protein kinases were coincubated with 10 μM FSBA with varied amounts of staurosporine (0.1 μM-10 μM) for 1 to 2 hours at room temperature.

Autoradiography and LC/MS were used to evaluate FSBA as an activity-based probe for protein kinases. The work presented here demonstrates FSBA's utility as an activity-based probe and that LC/MS can be used as a screening tool for the selection of ATP competitor protein kinase inhibitors.

### Example 1

### Affinity Labeling of FSBA Detected By Autoradiography

FSBA, an ATP analogue, is an affinity label that covalently labels most protein kinases by binding to the ATP pocket. For initial FSBA labeling studies, recombinantly expressed and purified kinase domain of the transforming growth factor (TGF)-β type I receptor (activin receptor-like kinase, ALK5; Laping et al., Mol. Pharmacol. 62:58-64 (2002)) was used. TGF-β acts through ALK5 to activate various mediators. Because TGF-β is a potent stimulus for extracellular matrix synthesis, inhibition of ALK5 activity may be beneficial in fibrotic disorders (Kanzler, et al., Am. J. Physiol. 39:G1059-G1068 (1999); Laping, et al. 2002).

¹⁴C-labeled FSBA was incubated with purified preparations of ALK5 (Laping et al., 2002) for one hour at room temperature. The samples were either heated to 95° C. or kept on ice for 10 minutes prior to the addition of ¹⁴C-FSBA. Samples were subjected to SDS-PAGE and were either processed for autoradiography or stained with Coomassie blue. Autoradiography results indicated that FSBA labeled unheated ALK5 and CDK2 but did not label heat-denatured ALK5. In cold competition experiments, cold FSBA competed with the binding of ¹⁴C-FSBA to ALK5 kinase. According to the Coomassie blue staining pattern of the unheated and heated samples, FSBA binds only to the native-like conformations of ALK5. Similar results were obtained for a panel of other kinases.

Earlier studies have shown that ATP competes with FSBA modification of many kinases (Kamps et al., 1984; Fox et al., 1999; T'Jampens et al., 2002). To extend these studies to ALK5, purified ALK5 was incubated for 1 hour with ˜20 μM ¹⁴C-FSBA and 1 mM or 10 mM ATP. The autoradiography and Coomassie blue staining of the gel indicated that most of the labeling was blocked by 1 mM ATP and 10 mM ATP blocked ¹⁴C-FSBA so autoradiograph did not detect ¹⁴C-FSBA binding. These results indicate that FSBA and ATP compete for the same binding pocket in ALK5. Together the heat denaturation and ATP protection results indicated that FSBA has potential to be an activity-based probe for kinase profiling studies.

### Example 2

### LC/MS as an Alternative Technique to Follow Affinity Labeling by FSBA

Time-dependent reactions were performed to assess FSBA modification of kinases by LC/MS. Purified ALK5 was incubated with FSBA at room temperature and aliquots of samples taken out at defined time intervals were subjected to LC/MS. Deconvulated mass spectrum profile revealed the time-dependent covalent modification of ALK5 with FSBA. Purified ALK5 gave a single predominant parent peak with a molecular mass of 34,974 Da, expected for the baculovirus-expressed unphosphorylated form of ALK5 protein (Laping, et al., 2002). Incubation in the presence of FSBA caused a shift in molecular mass to 35,405 Da in a time-dependent manner. By 60 minutes, the parent peak was converted to a new peak with increased mass of 433 Da. Likewise, the covalent modification of ALK4 and CDK2, two other recombinantly expressed and purified kinases was also completed in about 1 hour. This modification was demonstrated by an increase (433 Da) in the molecular mass of FSBA-treated ALK4 and CDK2 proteins.

Under the same conditions, FSBA did not modify trypsin and BSA (kinases that do not contain an ATP binding site), indicating FSBA's selective reactivity to ATP-requiring kinases tested under these studies. The difference in the unmodified and FSBA-modified forms of each of the kinases tested accounts for the presence of covalently attached sulfonyl benzoyl adenosine moiety with the removal of fluorine atom. These results are analogous to the affinity labeling of P38γ by FSBA as reported by Fox et al. (1999). Based on their LC/MS studies, both the unphosphorylated and the phosphorylated forms of P38γ were modified by FSBA as seen by increase in mass of 433 Da of the FSBA-treated protein.

### Example 3

### ATP Competes With FSBA Modification as Determined by LC/MS

Autoradiography studies showed that ATP competes with FSBA modification of ALK5 kinases. To follow ATP protection effect by LC/MS and to extend the ALK5 results to other kinases, purified ALK5, ALK4 and CDK2 kinases were coincubated in the presence of FSBA and varying amounts of ATP (0.1 μM-1 mM) as presented in Table 1.

TABLE 1
Effect of ATP and FSBA on Mass Peak of ALK5,
ALK4, and CDK2 measured by LC/MS

| Protein | | Mass Peak (Da) |
|---|---|---|
| Kinase | Additives | (relative percentage) |
| ALK5 | none | 34,972 Da (100%) |
| ALK5 | FSBA (10 μM) | 35,405 Da (100%) |
| ALK5 | FSBA (10 μM) plus ATP (50 μM) | 35,405 Da (100%) |
| ALK5 | FSBA (10 μM) plus ATP (100 μM) | 35,405 Da (70%), |
| | | 34,972 Da (30%) |
| ALK5 | FSBA (10 μM) plus ATP (500 μM) | 34,972 Da (100%) |
| ALK4 | none | 35,025 Da (100%) |
| ALK4 | FSBA (10 μM) | 35,459 Da (100%) |
| ALK4 | FSBA (10 μM) plus ATP (50 μM) | 35,459 Da (95%), |
| | | 35,025 Da (5%) |
| ALK4 | FSBA (10 μM) plus ATP (100 μM) | 35,459 Da (70%), |
| | | 35,025 Da (30%) |
| ALK4 | FSBA (10 μM) plus ATP (500 μM) | 35,025 Da (100%) |
| CDK2 | none | 33,971 Da (100%) |
| CDK2 | FSBA (10 μM) | 34,404 Da (100%) |
| CDK2 | FSBA (10 μM) plus ATP (1 mM) | 34,404 Da (80%), |
| | | 33,971 Da (20%) |
| CDK2 | FSBA (10 μM) plus ATP (5 mM) | 34,404 Da (10%), |
| | | 33,971 Da (90%) |
| CDK2 | FSBA (10 μM) plus ATP (10 mM) | 33,971 Da (100%) |

ATP protected covalent modification of both ALK5 and ALK4 by FSBA in a concentration-dependent manner; most of the covalent modification was inhibited in the presence of 0.5 mM ATP. Likewise ATP protected labeling of CDK2 with FSBA in a concentration-dependent manner, although 5 mM ATP was necessary to block FSBA binding. Similar results were obtained by autoradiography using ¹⁴C-FSBA. These results suggest that the affinity of ATP for ALK5 and ALK4 is significantly higher than that for CDK2. These results also suggest that LC/MS in combination with FSBA modification provides a rapid way to assess a compounds ability to bind the ATP binding pocket of a kinase.

### Example 4

### Staurosporine Competes With FSBA Labeling

ATP competitor small molecule protein kinase inhibitors, which also encompass the ATP binding pocket, were determined to prevent covalent modification of kinases by FSBA. Staurosporine, a microbial alkaloid, is a potent, but non-specific protein kinase inhibitor (Rueegg et al., Trends Pharmacol. Sci. 10:218 (1989); Garcia-Echeverria et al., 2000). It inhibits a number of kinases (Jacobson, et al., J. Cell Biol. 133:1041-1051 (1996); Schnier, et al., Proc. Natl. Acad. Sci. USA 1041-1051 (1996)), including CDK2 (DeBondt, et al., Nature 363:595-602 (1993); Lawrie, et al., Nature Structural Biology 4:796-801 (1997); Zhao, et al., J. Biol. Chem. 277:46609-46615 (2002)) and ALK5 (N. Laping, unpublished results), with IC₅₀ values in the nanomolar range. Crystal structures of CDK2 and Chk1 kinases complexed with staurosporine have been reported (Lawrie, et al., 1997; Zhao, et al., 2002). In both structures, staurosporine was shown to bind in the ATP binding cleft with the terahydropyran ring in a boat conformation.

To evaluate protective effects of staurosporine, purified ALK5, ALK4, CDK2, were coincubated with FSBA and increasing amounts of staurosporine. Samples were analyzed by LC/MS. LC/MS profiles of each of the kinases tested in the presence of FSBA and staurosporine demonstrated that staurosporine abrogated FSBA labeling in concentration-dependent manner. FSBA binding to all enzymes tested was essentially blocked by the addition of 10 μM staurosporine as shown in Table 2.

TABLE 2
FSBA and Staurosporine effects of Mass Peak of ALK5,
ALK4, and CDK2 measured by LC/MS

| Protein | | Mass Peak (Da) |
|---|---|---|
| Kinase | Additives | (relative percentage) |
| ALK5 | none | 34,972 Da (100%) |
| ALK5 | FSBA (10 μM) | 35,405 Da (100%) |
| ALK5 | FSBA (10 μM) plus staurosporine | 35,405 Da (90%), |
| | (0.1 μM) | 34,972 Da (10%) |
| ALK5 | FSBA (10 μM) plus staurosporine (1 μM) | 35,405 Da (70%), |
| | | 34,972 Da (30%) |
| ALK5 | FSBA (10 μM) plus staurosporine (10 μM) | 34,972 Da (100%) |
| ALK4 | none | 35,025 Da (100%) |
| ALK4 | FSBA (10 μM) | 35,459 Da (100%) |
| ALK4 | FSBA (10 μM) plus staurosporine | 35,459 Da (100%) |
| | (0.1 μM) | |
| ALK4 | FSBA (10 μM) plus staurosporine (1 μM) | 35,459 Da (80%), |
| | | 35,025 Da (20%) |
| ALK4 | FSBA (10 μM) plus staurosporine (10 μM) | 35,025 Da (100%) |
| CDK2 | none | 33,971 Da (100%) |
| CDK2 | FSBA (10 μM) | 33,404 Da (100%) |
| CDK2 | FSBA (10 μM) plus staurosporine | 33,404 Da (100%) |
| | (0.1 μM) | |
| CDK2 | FSBA (10 μM) plus staurosporine (1 μM) | 33,404 Da (70%), |
| | | 33,971 Da (30%) |
| CDK2 | FSBA (10 μM) plus staurosporine (10 μM) | 33,971 Da (100%) |

### Example 5

### Biotinylated FSBA (biotin-FSBA)

Based on the crystal structure of CHK1/FSBA complex, and the published studies of Scoggins, et al. (1996), FSBA was determined to support a biotin moiety at either the R₂ or R₃ position in its ribose moiety. Compounds of Formula I were synthesized

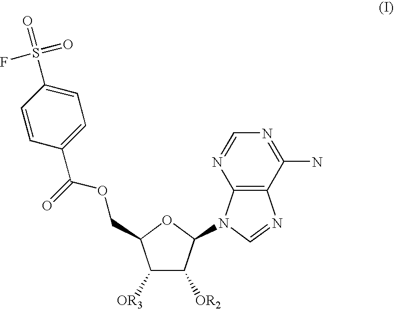

where
R₂ and R₃ are independently H or biotin as represented by Formula II:

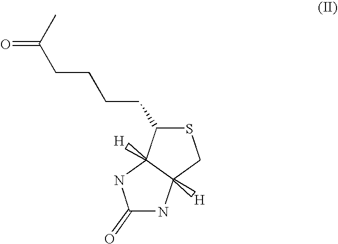

Compounds that were biotinylated at both the 3′ and the 2′ hydroxy groups were separated from FSBA comprising one biotin moiety using HPLC. 3′ and 2′ biotinylated FSBA were further separated from each other using HPLC. Each singly biotinylated FSBA has a molecular weight of approximately 680 Da.

Biotinylated FSBA may be produced as a free compound for use in solution or it may be attached to a solid support or resin.

### Example 6

### Synthesis of Biotin-FSBA

(+)-Biotin (0.070 g, 0.29 mmol) was dissolved in dry N,N-dimethylformamide (DMF, 1 mL) with heating. Upon cooling, di-isopropylcarbodiimide (25 μL, 0.16 mmol) was added and the mixture allowed to stand for 1 hour at room temperature. The partially gelled solution was then added to an ice-cold solution of 5′-(4-fluorosulphonylbenzoyl) adenosine. 1DMF (FSBA, 0.0563 g, 0.1 mmol) and diisopropylethylamine (17.4 μL, 0.1 mmol) in dry DMF (800 μL). After 5 minutes, a solution of N,N-dimethylaminopyridine (0.0122 g) in dry DMF (200 μL) was added and the mixture allowed to warm slowly to room temperature. The reaction was followed by analytical HPLC (Spherisorb S5 ODS2, 40% to 60% B over 45 minutes A=0.1% trifluoroacetic acid (TFA)/water, B=0.1% TFA/90% acetonitrile/10% water). The ratio of isomers produced during the reaction changed with time reaching equilibrium after overnight stirring.

The reaction mixture was evaporated to dryness, re-dissolved in the minimum volume of acetic acid/water/acetonitrile (10/40/50 by volume) and then purified by preparative HPLC (Hypersil 5u BDS C8, 250×21.2 mm, 25% to 50% B over 80 minutes). Fractions containing the 2′ (eluted first) and 3′ isomers (1) were pooled and then lyophilised (10.2 mg and 9.8 mg recovered respectively).

### Example 7

### Synthesis of Biotin-FSBA

(+)-Biotin (0.032 g, 0.143 mmol), 2-(1H-benzotriazole-1-yl)-1,1,3,3-tetramethyluronium hexafluorophosphate (0.054 g, 0.14 3 mmol), N-hydroxybenzotriazole.H₂O (0.022 g, 0.143 mmol), N,N-dimethylaminopyridine (0.0175 g, 0.143 mmol) and 5′-(4-fluorosulphonylbenzoyl) adenosine (FSBA, 0.0563 g, 0.1 mmol) were dissolved in dry N,N-dimethylformamide (0.5 mL) and chilled in an ice bath. N,N-diisopropylethylamine (0.075 mL, 0.43 mmol) was added and after allowing the mixture to warm to room temperature, was stirred overnight. The reaction was followed by analytical HPLC (Spherisorb S5 ODS2, 40% to 60% B over 45 minutes A=0.1% trifluoroacetic acid (TFA)/water, B=0.1% TFA/90% acetonitrile/10% water). The ratio of isomers produced during the reaction changed with time reaching equilibrium after overnight stirring.

The reaction mixture was evaporated to dryness, re-dissolved in the minimum volume of acetic acid/water /acetonitrile (10/40/50 by volume) and then purified by preparative HPLC (Hypersil 5u BDS C8, 250×21.2 mm, 25% to 50% B over 80 minutes). Fractions containing the 2′ (eluted first) and 3′ isomers (1) were pooled and then lyophilized (10.2 mg and 9.8 mg recovered respectively).

### Example 8

### Binding of Biotin-FSBA with ALK5

Purified ALK5 (10 μg) was incubated with 3′biotin-FSBA at 2 μM, 10 μM, and 20 μM at room temperature in separate containers. The contents of each container were subjected to LC/MS. Deconvulated mass spectrum profile revealed a concentration-dependent covalent modification of ALK5 with 3′biotin-FSBA. Purified ALK5 gave a single predominant peak with a molecular mass of 34,971 Da, expected for the baculovirus-expressed unphosphorylated form of ALK5 protein (Laping et al., 2002). Incubation in the presence of 3′biotin-FSBA caused a shift in molecular mass to 35,631 Da in a concentration-dependent manner. When 10 μg ALK5 was incubated with 20 μM 3′biotin-FSBA, the parent peak was converted to a new peak with increased mass of about 660 Da. Similar results were observed when CDK2 was incubated with 3′biotin-FSBA.

These results indicate that biotinylated FSBA modifies protein kinase as effectively as non-biotinylated FSBA. Similarly, these results indicate that LC/MS may be used to detect biotin-FSBA association with a kinase.

### Example 9

### ATP Competes With Biotin-FSBA Modification

ALK5 was incubated with and without biotin-FSBA (20 μM). Aliquots of ALK5 with biotin-FSBA were also incubated with ATP at the following concentrations: 0.1 mM, 0.5 mM, 1.0 mM, and 10 mM. In addition, aliquots of ALK5 and biotin-FSBA were incubated with staurosporine at the following concentrations: 0.1 μM, 1.0 μM, 5.0 μM, 10.0 μM, and 100.0 μM.

Samples were prepared for Western blot as follows. Aliquots of each sample were subjected to SDS-PAGE and transferred onto PVDF membranes by electroblotting. After transfer, the membranes were blocked in Tris-buffered saline (TBS) with 1% Tween (TBS-Tween) and 3% (wt/vol) nonfat dry milk for 1 hr at room temperature. Blots were then treated with an avidin-horseradish peroxidase conjugate (Bio-Rad, 1:1000 dilution) in TBS-Tween with 3% nonfat dry milk for 1 hour at room temperature. The blot was washed with TBS-Tween three times (5-10 min each wash) and then treated with Supersignal West Pico Chemiluminescent substrate (Pierce) for 1 minute. The blot was exposed and developed by using an AlphaImager 2000 (Alpha Innotech).

Coomassie stained SDS-PAGE and Western blot indicated that both ATP and staurosporine competed with biotin-FSBA for ALK5 binding. Purified ALK5 in the presence of biotin-FSBA was shifted on SDS-gel as compared with ALK5 alone indicating a higher molecular weight. In the presence of ATP (≧1.0 mM) or staurosporine (≧5.0 μM) biotin-FSBA/ALK5 bands were no longer visible as determined by Western blot.

### Example 10

### ALKS Inhibitor Blocks Binding of Biotin-FSBA to ALK5

Purified ALK5 (5 μg), CDK2 (10 μg) and BSA (5 μg) were each incubated with biotin-FSBA (40 μM) in separate containers at room temperature for about 2 hours. Samples were incubated with biotin FSBA alone and with a known ALK5 inhibitor (SB431542) at 20 μM. SB431,542 is described in as an ALK5 inhibitor in Gareth, et al., Mol Pharmacol (2002) 62: 65-7 and Laping, et al., Mol Pharmacol (2002) 62: 58-64. Samples were then run on SDS-PAGE gels and Coomassie staining and Western blot, as described in Example 9, was performed.

Biotin-FSBA was detected by Western blot to bind enzyme in samples of CDK2 with or without ALK5 inhibitor. Western blot showed no bands for samples of BSA indicating that biotin-FSBA did not bind to BSA. Samples containing ALK5 and biotin-FSBA were detected by Western blot, but no band was detected for the ALKS sample with inhibitor indicating that biotin-FSBA did not bind to ALK5 in the presence of ALK5-inhibitor.

### Example 11

### Profiling Kinases From Complex Proteome With Biotin-FSBA

Triton extracted HeLa cells were lysed and dialyzed to remove ATP and ADP. Cell lysate was pre-cleared with avidin beads. Cell extract was incubated with and without straurosporin (500 μM) for 2 hours at room temperature. Lysate samples were then incubated with biotin-FSBA for an additional 4 hours. Samples were run on SDS-gels and stained with Coomassie blue. Western blot was also performed, as described in Example 9. Coomassie stained gels indicted little difference in gel bands among samples. Western blot analysis indicated a dramatic decrease in band intensity for selected bands in samples where staurosporin was present, indicating that staurosporin competes with biotin-FSBA for certain proteins in the cell lysate.

### Example 12

### Profiling Kinases From Complex Proteome With Biotin-FSBA and Selective Inhibitors

HeLa cells were lysed pre-cleared as described in Example 9. Cell lysate (80 μg) was incubated in separate containers with no kinase inhibitor or with 500 μM staurosporin, or one of two selective inhibitors of Rho-associated protein kinase/ROCK (herein after “ROCK”) for 2 hours at room temperature. Biotin-FSBA was added (20 μM) to each container and incubated for another 4 hours at room temperature. Aliquots of each mixture were analyzed by Western Blot as described in Example 9. ROCK and selective inhibitors of ROCK are described in Uehata, et al., Nature (1997) 389:990-994.

Cell extract incubated with staurosporin or with one of two ROCK inhibitors showed reduced intensity in selective bands as compared with cell extract incubated with no inhibitors, indicating a reduced binding of biotin-FSBA to selective proteins in the presence of kinase inhibitors. This decrease in band intensity may be used to further identify potential kinases with the cell lysate.

### Example 13

### ROCK1 Inhibitors Block Binding of Biotin-FSBA to ROCK but Not to ALK5

Approximately 10 μg of human ROCK1 (hROCK1) (48 kDa) or ALK5 (34 kDa) were separately co-incubated with 10 μM FSBA with and without two known hROCK1 inhibitors (SB-742548 and Y-27632) for 3 hours at room temperature. Reactions were stopped with 0.1% TFA. Samples were analyzed by LC/MS. LC/MS profiles of each of the inhibitors tested demonstrated that while hROCK1 inhibitors abrogated FSBA labeling of ROCK1 they did not inhibit FSBA labeling of ALK5. The compound SB-742548 and similar compounds are described in WO 03/080610, which is herein incorporated by reference in its entirety. Y-27632 is described as a selective inhibitor of ROCK1 in Uehata, et al., Nature (1997) 389:990-994.

### Example 14

### Mode of Action of ROCK Inhibitors

Approximately 10 μg of human ROCK1 (hROCK1) (48 kDa) was co-incubated with 10 μM FSBA with and without Y-27632, at a 1:1 ratio with FSBA or a 1:5 concentration ratio with FSBA for 3 hours at room temperature. Reactions were stopped with 0.1% TFA. Samples were analyzed by LC/MS. LC/MS profiles demonstrated that Y-27632 inhibits hROCK1 at a 1:1 ratio. Thus, this inhibitor acts a competitive inhibitor of hROCK1.

### Example 15

### Evaluation of ROCK Inhibitors on ALK5

Purified ALK5 (5 μg) was incubated with FSBA (40 μM) with and without a ROCK inhibitor (Y-27632) for about 2 hours. Reactions were stopped with 0.1% TFA. Samples were analyzed by LC/MS. LC/MS profiles tested in the presence of FSBA demonstrated that Y-27632 had no effect on FSBA binding to ALK5.

The above description fully discloses how to make and use the present invention. However, this invention is not limited to the particular embodiments described hereinabove, but includes all modification thereof within the scope of the appended claims and their equivalents. Those skilled in the art will recognize through routine experimentation that various changes and modifications may be made without departing from the scope of this invention. The various references to journals, patents and other patent applications that are cited herein are incorporated by reference herein as though fully set forth.

We claim:

1. A method for identifying a compound that inhibits an enzyme having an ATP binding site comprising the steps of:
(a) contacting a composition comprising the enzyme having an ATP binding site, an analyte capable of binding to the ATP binding site of said enzyme, and a test compound, and
(b) detecting whether said test compound inhibits said analyte from binding said ATP binding site.

2. The method of claim 1, wherein said enzyme is a kinase.

3. The method of claim 1 wherein said test compound is a competitive inhibitor of said analyte.

4. The method of claim 1 wherein said analyte is p-flurosulfonylbenzoyl 5′-adenosine (FSBA).

5. The method of claim 4, wherein the FSBA is biotinylated.

6. The method of claim 1, wherein the enzyme comprises a conserved lysine in the ATP binding site.

7. The method of claim 6, further comprising binding the analyte to the conserved lysine.

8. The method of claim 1 wherein said detecting step comprises using mass spectrometry.

9. The method of claim 1, wherein said detecting step comprises using a protease assay.

10. The method of claim 1, wherein said detecting step comprises using a kinase assay.

11. The method of claim 1, wherein said detecting step comprises using Western blot.

12. A method for identifying a compound that inhibits a kinase having an ATP binding site comprising the steps of:
(a) contacting a composition comprising a kinase and an analyte that binds to an ATP binding site of said kinase,
(b) detecting binding of said analyte to said ATP binding site,
(c) contacting a composition comprising said kinase, said analyte, and a test compound, and
(d) detecting whether said test compound inhibits said analyte in step (c) from binding said ATP binding site.

13. The method of claim 12, wherein the kinase comprises a conserved lysine in the ATP binding site.

14. The method of claim 13, further comprising binding the analyte to the conserved lysine.

15. The method of claim 12, wherein said detecting step comprises using mass spectrometry.

16. The method of claim 12, wherein said detecting step comprises using a protease assay.

17. The method of claim 12, wherein said detecting step comprises using a kinase assay.

18. The method of claim 12, wherein said detecting step comprises using Western blot.

19. The method of claim 12, wherein said test compound is a competitive inhibitor of said analyte.

20. The method of claim 12, wherein said analyte is p-flurosulfonylbenzoyl 5′-adenosine (FSBA).

21. The method of claim 20 wherein the FSBA is biotinylated.

22. A method for identifying a test compound that inhibits a kinase having and ATP binding site comprising the steps of:
(a) contacting a composition comprising the kinase and test compound,
(b) contacting a composition comprising said kinase and said test compound with an analyte, and
(c) detecting whether said test compound inhibits said analyte in step (b) from binding said ATP binding site.

23. The method of claim 22, wherein the kinase comprises a conserved lysine in the AT? binding site.

24. The method of claim 23, further comprising binding the analyte to the conserved lysine.

25. The method of claim 22, wherein said detecting step comprises using mass spectrometry.

26. The method of claim 22, wherein said detecting step comprises using a protease assay.

27. The method of claim 22, wherein the said detecting step comprises using a kinase assay.

28. The method of claim 22, wherein said test compound is a competitive inhibitor of said analyte.

29. The method of claim 22, wherein said analyte is p-flurosulfonylbenzoyl 5′-adenosine (FSBA).

30. The method of claim 29, wherein said FSBA is biotinylated.

31. A method for identifying a protein kinase having an ATP binding site comprising the steps of:
(a) contacting a composition comprising the protein kinase with an analyte capable of bind said kinase, and
(b) detecting whether analyte binds to said kinase.

32. The method of claim 31, wherein said analyte is p-flurosulfonylbenzoyl 5′-adenosine (FSBA).

33. The method of claim 32, wherein the FSBA is biotinylated.

34. The method of claim 31, further comprising contacting said protein kinase with a kinase inhibitor.

35. The method of claim 31, wherein said detecting step comprises using Western blot.

36. The method of claim 31, wherein said detecting step comprises using LC/MS.

37. A compound of Formula I 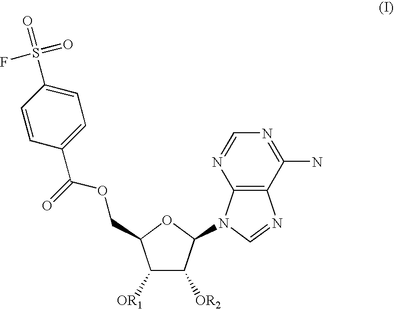
where
R₁ and R₂ are independently H or biotin as represented by Formula II:

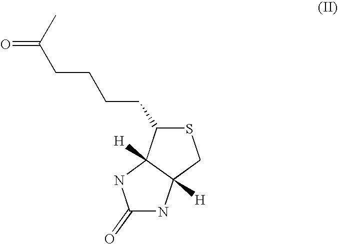

38. A process for making a compound of Formula I comprising the steps of:
a) dissolving (+)-Biotin, 2-(1H-benzotriazole-1-yl)-1,1,3,3-tetramethyluronium hexafluorophosphate in dry N,N-dimethylformamide and heating;
b) cooling the solution of step a);
c) adding di-isopropylcarbodiimide to the solution of step b); and
d) adding the solution of step c) to an ice-cold solution of 5′-(4-fluorosulphonylbenzoyl)adenosine 1DMF and diisopropylethylamine; and
e) adding N,N-dimethylaminopyridine in dry DMF to the solution of step d) and warming slowly.

39. A process for making a compound of Formula I comprising the steps of:
a) dissolving (+)-Biotin, 2-(1H-benzotriazole-1-yl)-1,1,3,3-tetramethyluronium hexafluorophosphate, N-hydroxybenzotriazole.H₂O, N,N-dimethylaminopyridine and 5′-(4-fluorosulphonylbenzoyl)adenosine in dry N,N-dimethylformamide;
b) chilling the solution of step a);
c) adding N,N-diisopropylethylamine to the solution of step b); and
d) warming the solution of step c) to room temperature and stirring;

40. A method for identifying the mode of action of a test compound that inhibits a first enzyme having a first ATP binding site comprising the steps of:
(a) contacting a composition comprising the first enzyme having a fist ATP binding site, an analyte capable of binding to the first ATP binding site of said enzyme, and a test compound, wherein the ratio of concentration of analyte to test compound is at least 1:1; and
(b) detecting whether said test compound inhibits said analyte from binding said first ATP binding site, wherein diminution of analyte binding to the first ATP binding site indicates competitive inhibition by said test compound.

41. The method of claim 40, further comprising:
(a) contacting a composition comprising a second enzyme having a second ATP binding site, an analyte capable of binding to the second ATP binding site of said second enzyme, and said test compound;
(b) detecting whether said test compound inhibits said an analyte from binding said second ATP binding site; and
(c) determining if said test compound is a selective inhibitor of said enzyme.

42. The method of claim 41, wherein said test compound that diminishes binding of said analyte to said first enzyme but does not diminish binding of said an analyte to said second enzyme is a selective inhibitor of said first enzyme.
